(19) United States
(12) Patent Application Publication
(10) Pub. No.: US 2020/0113489 A1
(43) Pub. Date: Apr. 16, 2020

(54) APPARATUS, SYSTEM AND METHOD FOR A MOTION SENSOR

(71) Applicant: MindMaze Holding SA, Lausanne (CH)

(72) Inventors: Benoit MARIANI, Lausanne (CH); Farzin DADASHI, Lausanne (CH); Stephane Lovejoy, Lausanne (CH)

(21) Appl. No.: 16/394,072

(22) Filed: Apr. 25, 2019

Related U.S. Application Data
(60) Provisional application No. 62/663,944, filed on Apr. 27, 2018.

(51) Int. Cl. A61B 5/11; G16H 40/67; G16H 20/30; A61B 5/00

(52) U.S. Cl. CPC A61B 5/1118; G16H 40/67; G16H 20/30; A61B 2560/0209; A61B 5/7425; A61B 5/0077; A61B 2562/0219; A61B 5/681

(57) ABSTRACT

A system, method and apparatus that is capable of automatically detecting and classifying various physical activities of a user. This enables such activities to be analyzed, for example according to the complexity of the activity and the amount of time spent in each activity. A barcode may be calculated, according to the various activities of the user, the amount of time spent in each activity and optionally also the complexity of each such activity.

### FIELD OF THE INVENTION

The present invention, in at least some embodiments, relates to a system, method and apparatus for activity classification for a watch sensor, and in particular to such a system, method and apparatus which can automatically detect and differentiate between various daily physical activities of the user for such a sensor.

### BACKGROUND OF THE INVENTION

Activity trackers, such as the Fitbit device or sports watches, track various physical activities of the user. However they are limited in their ability to provide a comprehensive picture of the user's activities, as these devices must be manually set by the user to track each activity relevant information.

### BRIEF SUMMARY OF THE INVENTION

The present invention, in at least some embodiments, provides a system, method and apparatus that is capable of automatically detecting and classifying various physical activities of a user. This enables such activities to be analyzed, for example according to the complexity of the activity and the amount of time spent in each activity. A barcode may be calculated, according to the various activities of the user, the amount of time spent in each activity, and the chronological occurrence of activities. The complexity score is a single number score which combines and interprets a plurality of trends to detect the initiation and/or continuation of chronic conditions, depression, or more healthy behavior, such as performing body movements or other activities that are deemed to be healthy. This complexity score is calculated by using the barcode of activity as the input.

The complexity score is a functional score that supports personalized recommendations. For example, such suggestions or recommendations may include but are not limited to breaking up long sitting periods and taking a walk; and/or while walking, trying to take a longer path and increasing the speed on the way to the destination. Even such small changes provide much more variety in physical activities, and thereby allows the person to adopt and maintain more complex physical behaviors. The higher complexity score one manages to maintain at old age, the better equipped one is to meet daily challenges, even at advancing ages.

Preferably, the apparatus comprises a combination of a wearable sensor and a computational device that is separate from the wearable sensor, such as a cellular telephone of the user for example. The wearable sensor preferably comprises an IMU, which may for example be added to an existing device, such as a watch or a wristband of a watch. By existing device or existing wearable, it is meant a wearable or device which does not have a primary purpose of attaching the sensor to the user. As a non-limiting example, if the existing device is a watch or a portion or accessory thereof (such as a wristband for example), the primary purpose of the watch is to tell time. The sensor may be added to the existing device during manufacturing or after manufacturing, and/or may be integrally formed with the existing device.

The apparatus further comprises software for analyzing the activities of the user. The apparatus is preferably in communication with a server, for example to store information in a database or to receive information regarding user activity classification history in order to provide pertinent coaching messages to the user.

Unless otherwise defined, all technical and scientific terms used herein have the same meaning as commonly understood by one of ordinary skill in the art to which this invention belongs. The materials, methods, and examples provided herein are illustrative only and not intended to be limiting.

Implementation of the apparatuses, devices, methods, and systems of the present disclosure involve performing or completing specific selected tasks or steps manually, automatically, or a combination thereof. Specifically, several selected steps can be implemented by hardware or by software on an operating system, of a firmware, and/or a combination thereof. For example, as hardware, selected steps of at least some embodiments of the disclosure can be implemented as a chip or circuit (e.g., ASIC). As software, selected steps of at least some embodiments of the disclosure can be performed as a number of software instructions being executed by a computer (e.g., a processor of the computer) using an operating system. In any case, selected steps of methods of at least some embodiments of the disclosure can be described as being performed by a processor, such as a computing platform for executing a plurality of instructions.

Software (e.g., an application, computer instructions) which is configured to perform (or cause to be performed) specific functionality may also be referred to as a “module” for performing that functionality, and also may be referred to a “processor” for performing such functionality. Thus, processor, according to some embodiments, may be a hardware component, or, according to some embodiments, a software component.

Further to this end, in some embodiments: a processor may also be referred to as a module; in some embodiments, a processor may comprise one or more modules; in some embodiments, a module may comprise computer instructions—which can be a set of instructions, an application, software—which are operable on a computational device (e.g., a processor) to cause the computational device to conduct and/or achieve one or more specific functionality. Furthermore, the phrase “abstraction layer” or “abstraction interface,” as used with some embodiments, can refer to computer instructions (which can be a set of instructions, an application, software) which are operable on a computational device (as noted, e.g., a processor) to cause the computational device to conduct and/or achieve one or more specific functionality. The abstraction layer may also be a circuit (e.g., an ASIC) to conduct and/or achieve one or more specific functionality. Thus, for some embodiments, and claims which correspond to such embodiments, the noted feature/functionality can be described/claimed in a number of ways (e.g., abstraction layer, computational device, processor, module, software, application, computer instructions, and the like).

Some embodiments are described concerning a “computer,” a “computer network,” and/or a “computer operational on a computer network.” It is noted that any device featuring a processor (which may be referred to as “data processor”; “pre-processor” may also be referred to as “processor”) and the ability to execute one or more instructions may be described as a computer, a computational device, and a processor (e.g., see above), including but not limited to a personal computer (PC), a server, a cellular telephone, an IP telephone, a smart phone, a PDA (personal digital assistant), a thin client, a mobile communication device, a smart watch, head mounted display or other wearable that is able to communicate externally, a virtual or cloud based processor, a pager, and/or a similar device. Two or more of such devices in communication with each other may be a “computer network.”

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in order to provide what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. In the drawings:

FIGS. 1A-1B show various non-limiting, exemplary systems according to at least some embodiments of the present invention, for classifying a physical activity of a user;

FIG. 2 shows a non-limiting, exemplary method for classifying an activity of a user;

FIG. 3 shows an exemplary, non-limiting implementation of a sensor that is added to an existing wearable;

FIG. 4 shows a non-limiting, exemplary flow for operating the watch of FIG. 3 with a data display;

FIGS. 5A and 5B show non-limiting, exemplary displays for use with the user device and/or wearable device described herein; and

FIG. 6 shows a non-limiting, exemplary flow for analyzing data from a sensor that is added to an existing wearable.

### DESCRIPTION OF AT LEAST SOME EMBODIMENTS

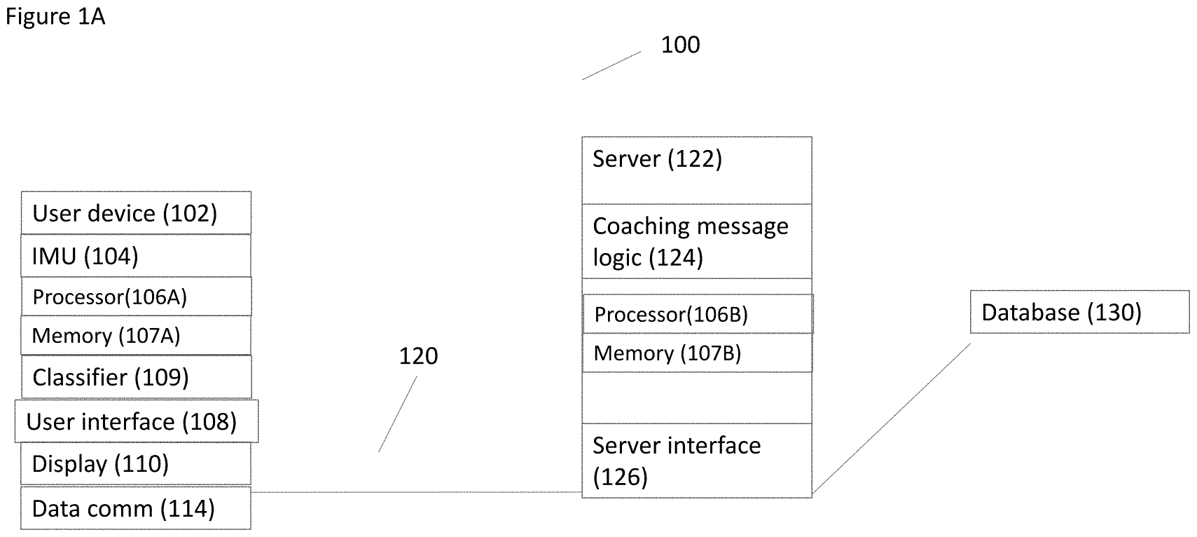

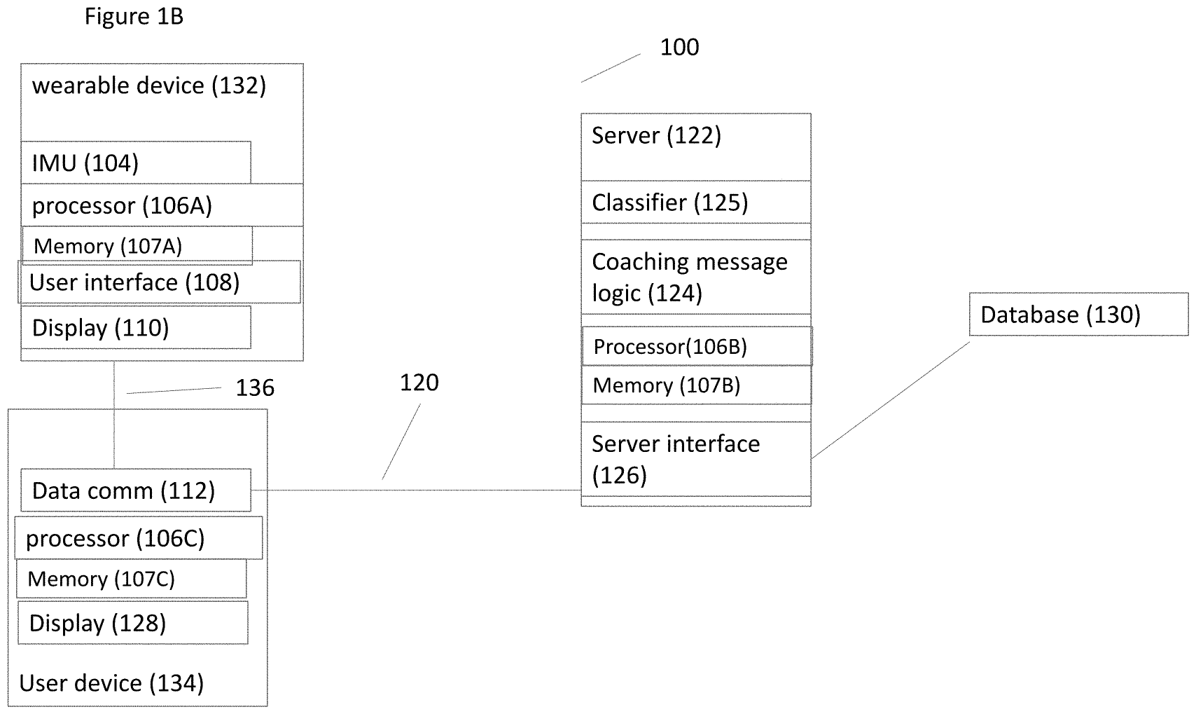

Turning now to the drawings, FIGS. 1A-1B show two non-limiting, exemplary systems according to at least some embodiments of the present invention, for classifying a physical activity of a user.

As shown, FIG. 1A shows a system 100, featuring a user device 102. In this non-limiting example, user device 102 is assumed to be a mobile communications device, such as a cellular telephone for example. User device 102 provides platform for graphical representation of activity feedback to the user as well as temporary data storage. However, user device 102 is preferably in communication with a remote server 122 through a mobile network 120 as shown, for additional services, and optionally to access and/or share additional data. Mobile network 120 may optionally comprise the Internet for example. The user is assumed to be holding, wearing or otherwise to be attached to user device 102, such that movements of the user are reflected in movements of user device 102.

User device 102 features an IMU (inertial measurement unit) 104 for collecting angular velocity and linear acceleration data, in regard to movements of user device 102, thereby collecting such data about movements of the user. IMU 104 is preferably selected for a suitable sensitivity and range, according to the functions described in greater detail below.

A processor 106A preferably receives such data from IMU 104. Preferably processor 106A is implemented as a microprocessor. Processor 106A executes instructions as stored in a memory 107A. Preferably server 122 also preferably features a processor 106B and a memory 107B. As used herein, a processor such as processor 107A or 107B generally refers to a device or combination of devices having circuitry used for implementing the communication and/or logic functions of a particular system. For example, a processor may include a digital signal processor device, a microprocessor device, and various analog-to-digital converters, digital-to-analog converters, and other support circuits and/or combinations of the foregoing. Control and signal processing functions of the system are allocated between these processing devices according to their respective capabilities. The processor may further include functionality to operate one or more software programs based on computer-executable program code thereof, which may be stored in a memory, such as memory 107A or 107B in this non-limiting example. As the phrase is used herein, the processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

A classifier 109, operated by processor 106A according to instructions stored in memory 107A, then classifies an activity of the user according to such data. As described in greater detail below, such activity classification preferably features selecting a category of such activity, such as for example sitting, standing, walking, running, gym and swimming. The activity classification result may optionally be transformed into a barcode, which displays both the length of time and sequential order of the user's activities. Therefrom, behavioral signature of the user can be extracted, preferably under form of complexity of the user's activities overall. Complexity computation involves time series entropy analysis.

The results of such classification, optionally including the barcode, may be displayed on a display 110, which may be integrally formed with or attached to user device 102. User device 102 also preferably features a user interface 108, which is displayed on display 110. Preferably, the results of the classification, as well as other visual information, is displayed through user interface 108 by display 110. User interface 108 also preferably accepts user commands, as display 110 is preferably a touchscreen. For example, the user may optionally select which data is to be displayed and for which time period.

Alternatively, display 110 is separate from a user interface 108. In this non-limiting example, user interface 108 may be provided through user device 102, such as through the touchscreen of a smart phone. Display 110 features an augmented reality display, in which the user manipulates or moves user device 102 to invoke the augmented reality display. For example, the user may move user device 102 so that a camera (not shown) that is attached to or otherwise associated with user device 102 is able to view a particular surface. A non-limiting example is provided below of such a surface being a watch portion or accessory, such as a watchband for example. Upon detecting such a surface, user device 102 then provides an augmented reality display of the information regarding the user's activities, as described in greater detail below.

User device 102 also preferably features a data communication module 112, for communicating with a server interface 126 of server 122 through computer network 120 as previously described. Data from IMU 104 and/or analysis results from analyzer 106 may be shared with server 122 through such communication. Such data and/or analysis results then may optionally be stored in a database 130. Optionally such data and/or analysis results are shared through a social sharing platform, including but not limited to Facebook, Twitter, Snapchat, Whatsapp and the like.

Server 122 may also optionally feature a coaching message algorithm 124, for providing suitable advice on type and duration of daily activity in order to improve activity behavior of the user.

FIG. 1B shows an alternative configuration of the system of FIG. 1A, in which various functions performed by the user device of FIG. 1A are instead performed by a wearable device 132. Wearable device 132 may optionally comprise a wristwatch, wristband or other wearable device that is worn by the user. Wearable device 132 comprises IMU 104, microprocessor 106, user interface 108 and display 110, with the same or similar functions as described with regard to the user device of FIG. 1A.

A user device 134 is optionally in communication with wearable device 132, for example through a wired or wireless connection 136. Such communication may for example enable the user to view the data on user device 134, through a display (not shown) or to perform various functions with regard to wearable device 132. In this non-limiting example, user device 134 may be a mobile device, such as a cellular telephone for example. User device 134 preferably comprises a processor 106C and a memory 107C, with functions as described above for example. A classifier 125 may operate on server 122. The functions of classifier 125 may be the same or similar to those of the classifier as described in FIG. 1A. The classifier may also be operated by the wearable device (not shown).

Server 122 may also feature a coaching message algorithm 124 as previously described.

Optionally user device 134 supports communication between wearable device 132 and server 122 as previously described, through data communication module 112. Alternatively, wearable device 132 communicates directly with server 122 (not shown).

Within either the user device or the wearable device, optionally the following components are included as a non-limiting implementation example:
Input 3D Acc @32 Hz only
Input 3D Angular velocity @50 Hz at least
Embedded C library with minimal footprint (12 kb allocated memory for execution on Nucleo F4)

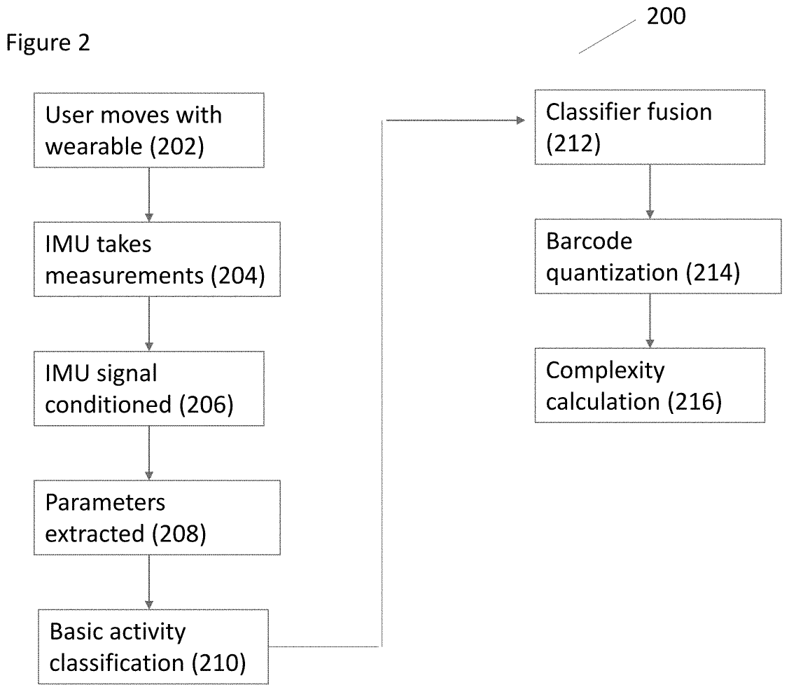

FIG. 2 shows a non-limiting, exemplary method for classifying an activity of a user. The method may optionally be performed with any of the systems of FIGS. 1A-1C. As shown, a method 200 begins with the user moving with a wearable device in stage 202. Although reference is made to a “wearable device”, optionally the user device of FIGS. 1A and 1C could also be used.

The IMU takes measurements as the user moves in stage 204. In stage 206, the IMU signals are conditioned. Such signal conditioning preferably includes performing a dynamic calibration is performed so IMU axes are virtually aligned to the functional movement axes. The calibration is preferably performed as an optimization that minimizes the difference between virtually-rotated-IMU signal and the function axis of body segments. Such a calibration means that the analyzer is able to determine the activity parameters without requiring specific direction of attachment of IMU to the user body.

In stage 208, various parameters are extracted, with regard to biomechanical parameters. This stage features the application of signal processing methods to extract information about duration of movement, interpretation of intensity, calculation of velocity and IMU orientation in 3D space. Optionally, in this stage, the method is based on extraction of cycle by cycle statistical features. The feature extraction method at this stage is insusceptible to cycle duration or amplitude and mainly dependent on geometric shape of the IMU signal at each cycle.

In stage 210, activity classification is performed, or at least a portion of such classification is performed. Once the IMU is aligned to the bodily axis (signal conditioning, from stage 204) and movement generic parameters are extracted, then the activity type can be classified.

Preferably, as shown, activity classification is performed in two stages: stage 210, in which a basic activity classification is performed; and stage 212, in which classifier fusion is performed. In this implementation, the activity labeling is performed via a hybrid classification at the two stages.

In stage 210, classification is performed, which provides a label for the type of activity and a confidence interval on the certainty of chosen label. For example, the classification may be performed according to multi-class QDA (quadratic discriminant analysis), a technique which is well known in the art. The features used for the covariance matrix of the QDA preferably include, but are not limited to, statistical features such as signal amplitude, auto-regressive coefficients that describe each cycle of IMU data (preferably in 6 channels), and the dynamic time warping cost.

In stage 212, classifier fusion is performed, based on the output of the 210 classifier and results obtained from performing dynamic time warping, to account for temporal effects.

In stage 214, barcode quantization is then performed. By determining physical activity type, duration, intensity and sequence a barcode can be calculated. Each physical activity has continuous intensity range which imposes curse of dimensionality for a later stage of calculating the complexity. In order to reduce the noise as well as complexity computational cost, the physical activity intensity is preferably quantized based on an optimization process.

The barcode optionally includes the following parameters in regard to each of the user's physical activities or groups of activities:

1. Type: lying, sitting, standing, . . .

2. Duration: sit-stand duration, sedentary vs. active periods

3. Intensity: Acceleration, velocity of movement, cadence

4. Pattern: Temporal sequence of activity types

5. Context: Indoor vs outdoor

Some non-limiting examples of the activity types and exemplary metrics that can be measured are given below:
Activity classes (Metrics shown in italics):
   Resting (duration)
   Sitting (duration)
   Standing still (sway jerkiness)
   . . . random movements (duration)
   Walking (steps, cadence, speed)
   Running (steps, cadence, speed)
   Gym
   Swimming

Once activity type, duration, intensity and sequence is determined, the temporal sequence of different physical activities is visualized as a barcode. The structural complexity of this barcode characterizes pain/frailty related physical activity and behavior of individuals. Physical activity quantification based on the above characteristics is also a key tool for user energy expenditure assessment.

In stage 216, complexity calculation is then performed. Entropy measures have been used to estimate the amount of “complexity” in a physiological system. A behavior with a greater degree of dynamical complexity shows higher entropy. Existing complexity metrics are based on a single time scale that limits the scope of interpretation to only that level and does not fully capture the dynamics of the entire system. The barcode complexity can be represented at multiple time scales using multi-scale-entropy (MSE). which allows to determine specific time scales at which pain/movement deficits occurs.

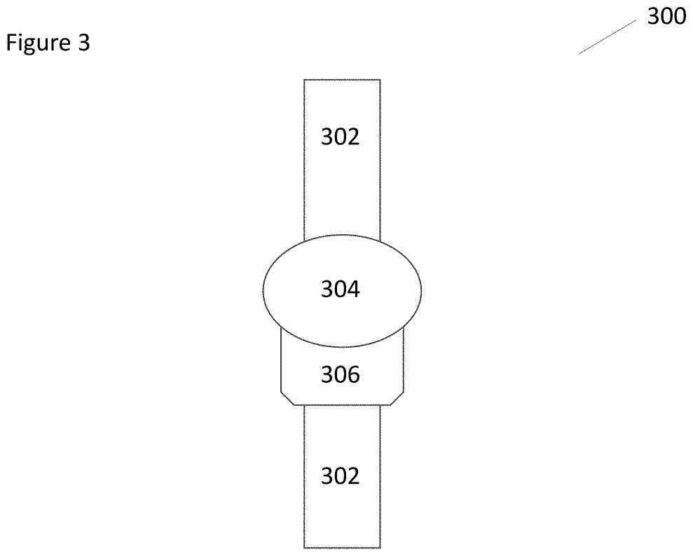

FIG. 3 shows an exemplary, non-limiting implementation of a sensor that is added to an existing wearable. The sensor is preferably attached to, or integrally formed with, a watch or a portion or accessory thereof, such as a watchband for example. For example the sensor could be encased in the watchband or strap, for example where the band or strap attaches to the watch body. Optionally the electronics of the analysis portion of the apparatus are incorporated to a portion of the watch or an accessory thereof.

A watch 300 features a watchband 302 and a timekeeping portion 304. A sensor module 306 preferably features the sensor and electronics for processing the signals from the sensor. The sensor preferably comprises an accelerometer for measuring acceleration, and optionally a gyroscope for measuring orientation. Optionally the sensor comprises an IMU as previously described. The accelerometer preferably has a processor, in addition to the processor of the electronics that process the sensor signals.

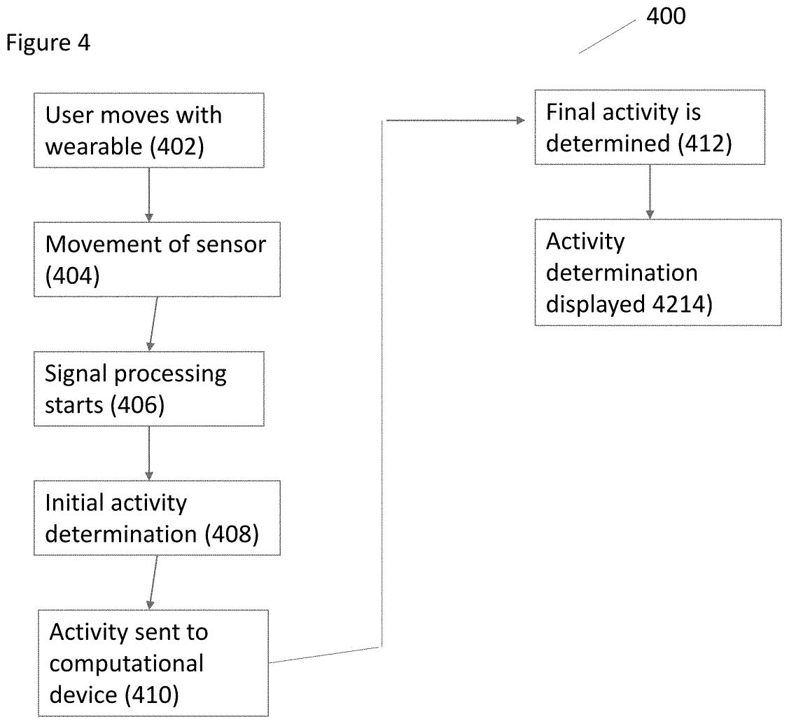

FIG. 4 shows a non-limiting, exemplary flow for operating the watch of FIG. 3 with a data display. In this non-limiting example, the data display is performed through augmented reality with a portable computational device, such as a cellular telephone for example, but the data display could also be performed in other ways. For example and without limitation, the data display could be performed through the watch or watchband without the portable computational device. Alternatively, the display could be performed through the portable computational device without the use of augmented reality.

Turning now to FIG. 4, a flow 400 begins with the wearer of the watch (also termed herein the user) performing an activity, such as walking for example, in 402. Such activity results in movement of the watch and hence of the sensor in 404. Such movement preferably causes the electronics of the sensor module to wake up and to start processing the signals from the sensor in 406. The signal processing leads to an initial activity determination being performed by the sensor module in 408, although alternatively such an initial activity determination is performed by the portable computational device. The initial activity determination or alternatively the processed signals are transmitted from the sensor module to the portable computational device in 410. Next, if the initial activity determination was performed by the sensor module, optionally a final activity determination is performed by the portable computational device in 412. Alternatively, if the initial activity determination was not performed by the sensor module, then an activity determination is performed by the portable computational device. The activity determination preferably includes an identification of the activity (walking, running, standing and so forth), a time that the activity was performed and a length of time over which the activity was performed.

In 414, the activity determination is displayed to the user, preferably through augmented reality. For example, the user could hold the portable computational device over a portion of the watch, which would then cause the activity determination to appear to be displayed on or by that portion of the watch.

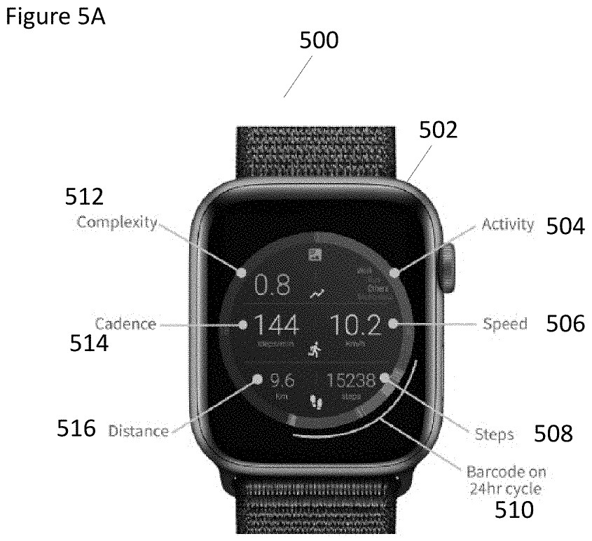

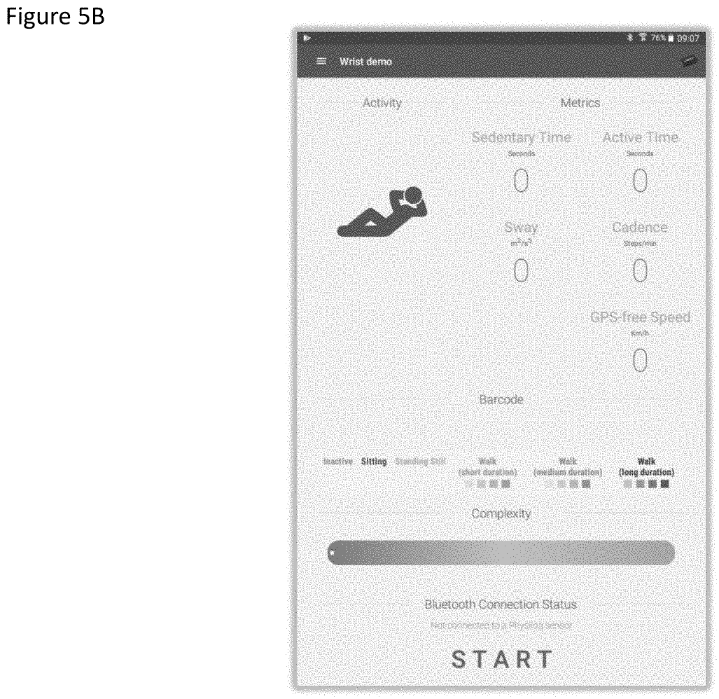

FIGS. 5A and 5B show non-limiting, exemplary displays for use with the user device and/or wearable device described herein. As shown in FIG. 5A, a watch 500 comprises a watch face 502, displaying such information as an activity 504 (for example that is currently being performed), a speed 506 of the activity, daily step count totals 508, a barcode of at least a plurality, if not all activities over 24 hours (shown as 510) and so forth. Complexity 512, cadence 514 and/or distance 516 of the current activity may also be shown.

FIG. 5B shows an exemplary, non-limiting app display for providing the above information.

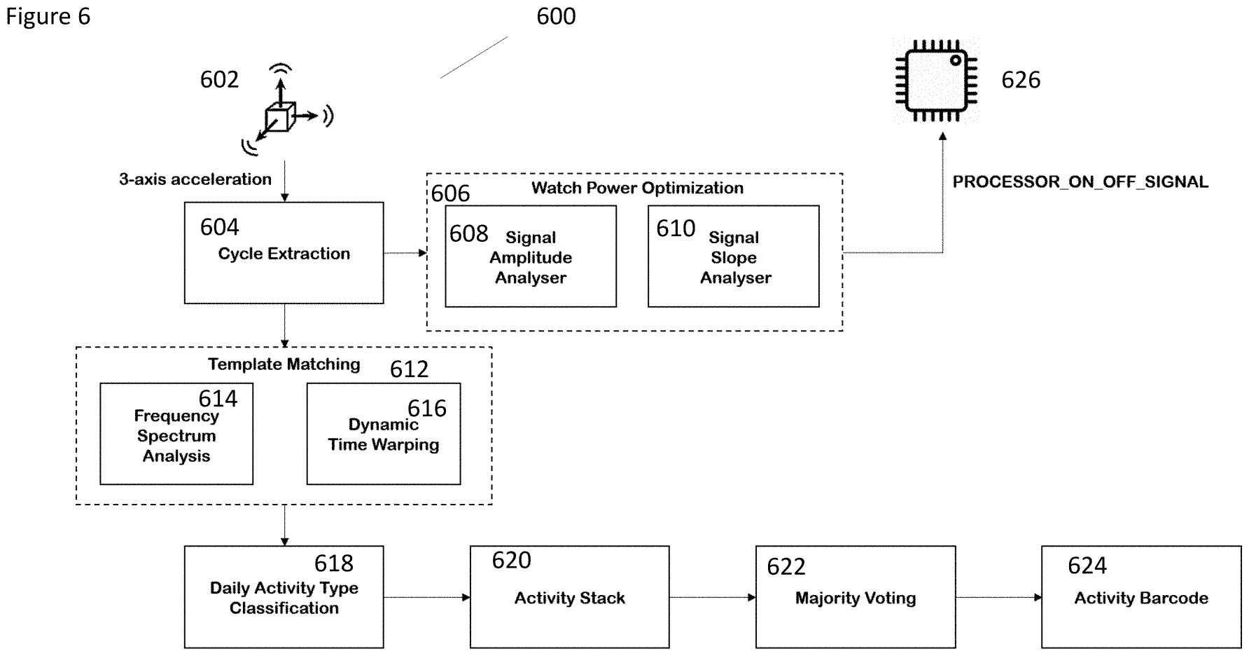

FIG. 6 shows a non-limiting, exemplary flow for analyzing data from a sensor that is added to an existing wearable. In a flow 600, a sensor 602 provides data. Sensor 602 preferably comprises an accelerometer for measuring acceleration, and optionally a gyroscope for measuring orientation. Optionally sensor 602 comprises an IMU as previously described. The accelerometer has a processor. The acceleration data can be used to determine the acceleration of the user, as sensor 602 is mounted in a known location, such as a watch or portion thereof for example.

Next in 604, cycle extraction is performed, to extract various parameters, with regard to biomechanical parameters. This stage features the application of signal processing methods to extract information about duration of movement, interpretation of intensity and acceleration. If orientation is being measured, optionally also calculation of velocity and IMU orientation in 3D space is performed. Optionally, in this stage, the method is based on extraction of cycle by cycle statistical features for frequency analysis.

At 606, watch power optimization is performed, to determine how frequently sensor 602 and the corresponding electronics are activated. As part of the optimization process, preferably a signal amplitude analyzer 608 determines an amplitude of the signal from sensor 602. A signal slope analyzer 610 determines a slope of the signal from sensor 602. According to the strength and noise level of the amplitude and slope, information is fed to a processor on/off signal 626, to determine whether sensor 602 is moving and so should be activated more or less frequently. The slope provides a more robust detection of activity or non-activity, as opposed to determining only the amplitude, which could be more susceptible to noise. The presence of a signal indicates movement, in order to wake up the electronics of the sensor data processing only when sensor 602 is moving.

In addition, the parameters from cycle extraction in 604 are fed to a template matching process 612, to compare the derived parameters to known patterns of various activities as previously described. Preferably the derived parameters are compared to known intensity patterns, to be able to estimate the relative intensity of the activity being engaged in. Determining such parameters over a period of time also enables a length of time over which the activity is being performed to be determined. The template matching process 612 may include frequency spectrum analysis 614, which relates to the previously described probabilistic analysis of the extracted parameters and which may be used for pattern matching between signals for features in the frequency domain. The template matching process 612 may also include a dynamic time warping process 616, which is used to find similarity between signals, to determine the matching between patterns for features in the time domain. The closest template or matched pattern is used to select the activity that is being performed.

Next a daily activity type classification process 618 is performed as previously described. A stack of such activities is determined in 620, for resolution over a period of time. Activities may be determined for a short period of time, such as for microseconds to seconds for example, and then stacked for a longer period of time, such as 1 minute or multiple minutes for example. Majority voting is used in 622 to determine which activity classifications are correct, given that there is a stack of a plurality of activity classifications, and also the start and end time of each such activity. These stacking and majority voting processes may also reduce power consumption by requiring a lower amount of data transmission to an accompanying apparatus, for example by Bluetooth. In 624, the activity barcode is determined as previously described.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. An apparatus for automatically detecting and classifying physical activities of a user, comprising an IMU and software for analyzing the activities of the user, wherein said IMU is implemented to be worn, held by or attached to the user in an existing device; wherein said existing device comprises a watch, a portion thereof and/or an accessory thereto.

2. The apparatus of claim 1, further comprising a cellular telephone for analyzing the activities of the user.

3. The apparatus of claim 1, wherein said existing device comprises a watchband.

4. A system comprising the apparatus of claim 3 and a user computational device, said user computational device comprising a display, a memory and a processor, wherein said memory stores instructions for receiving the analysis of the activities of the user and for causing an augmented reality display to be displayed by said display, said augmented reality display displaying information about the activities of the user, said instructions being executed by said processor.

5. The system of claim 4, in which the user manipulates or moves said user computational device to invoke the augmented reality display.

6. The system of claim 5, wherein said user computational device further comprises a camera and wherein said camera is manipulated to capture an image of said existing device, after which said augmented reality display is invoked.

7. The system of claim 4, wherein said instructions include instructions for watch power optimization, to determine how frequently the apparatus is activated.

8. The system of claim 7, wherein said apparatus further comprises a signal amplitude analyzer for determining an amplitude of the signal from said IMU and a signal slope analyzer for determining a slope of the signal from said IMU, to determine how frequently the apparatus is activated.

9. The system of claim 8, wherein said processor of said apparatus is turned off or on according to the strength and noise level of the amplitude and slope.

10. The system of claim 9, further comprising a server in communication with said apparatus, said server further comprising a database.

11. A method for analyzing physical activities of a user with the system of claim 4, comprising automatically detecting a category of physical activity of a user according to signals from the IMU.

12. The method of claim 11, further comprising automatically determining an amount of time spent in each activity.

13. The method of claim 12, further comprising automatically determining a complexity of each activity.

14. The method of claim 13, calculating a barcode of the physical activities of the user.
